(19) United States
(12) Patent Application Publication
(10) Pub. No.: US 2020/0231466 A1
(43) Pub. Date: Jul. 23, 2020

(54) INTELLIGENT SYSTEMS AND METHODS FOR PROCESS AND ASSET HEALTH DIAGNOSIS, ANOMOLY DETECTION AND CONTROL IN WASTEWATER TREATMENT PLANTS OR DRINKING WATER PLANTS

(71) Applicants: Zijun XIA, Pudong, Shanghai (CN); BL TECHNOLOGIES, INC., Minnetonka, MN (US)

(72) Inventors: Su LU, Shanghai (CN); Zijun XIA, Shanghai (CN); Zhaoyang WAN, Trevose, PA (US); Yu WANG, Shanghai (CN); Xijing BI, Shanghai (CN); Guoliang WANG, Shanghai (CN); Chuanyou TANG, Shanghai (CN); Zhiping ZHU, Shanghai (CN); Wenchao MA, Beijing (CN); Qin DONG, Pudong Shanghai (CN); Sijing WANG, Shanghai (CN); Yisong LI, Shanghai (CN); Jiajia LING, Shanghai (CN)

(21) Appl. No.: 16/472,998

(22) PCT Filed: Oct. 9, 2017
(86) PCT No.: PCT/CN2017/105377

§ 371 Date: Jun. 24, 2019

(51) Int. Cl. C02F 1/00; G06N 3/08; G06N 5/04; G06N 20/10; C02F 3/00; G01N 33/18

(52) U.S. Cl. CPC C02F 1/008; C02F 2209/22; G06N 5/04; G06N 20/10; C02F 3/006; G01N 33/18; C02F 2209/02; C02F 2209/40; C02F 2209/06; C02F 2209/08; C02F 2209/07; C02F 2209/14; C02F 2209/15; C02F 2209/16; C02F 2209/18; C02F 2209/38; G06N 3/088

(57) ABSTRACT

Described herein are systems and methods of analyzing data acquired from a water plant, both historical and in real-time, making determinations about process and asset health diagnosis and anomaly detection using advanced techniques, and controlling the plant and/or providing alerts based on such determinations.

### FIELD OF THE INVENTION

Disclosed herein are systems and methods of analyzing data acquired from a water plant, both historical and in real-time, making determinations about process and asset health diagnosis and anomaly detection using advanced techniques, and controlling the plant and/or providing alerts based on such determinations.

### BACKGROUND

Waste water treatment plants and drinking water plants need daily monitoring and operation to ensure the process health to meet the effluent standards and lower the operation cost at the same time. Treatment process diagnosis, data anomaly identification, equipment health diagnosis are key steps for operators to make the correct decisions or control actions. Traditionally, water treatment is a long process with large volumes of data generated from sensors or lab tests such as water quality sensors and assets sensors. Currently, most of the daily diagnosis is made by human based on experience and simple data analysis such as threshold judgement. It is difficult to handle multi-parameters at the same time to analyze the possible sensor fraud or health issues to make the best control all the time. Different people making such decisions and judgments may result in different quality levels of water plant management. Furthermore, large margins are kept during plant design and operations based on experience to make sure the effluent standard is met even under the worst case, which leads to much higher operation cost. An intelligent diagnostic system can help people improve efficiency in daily operation and improve the quality of diagnosis which is comprehensive and reliable. Such a system could also help to improve the operation quality, prevent the failures timely and ultimately increase the benefits.

Therefore, a method and system is desired to quickly, continuously and accurately diagnose process and asset health, detect anomalies, and dynamically control the water treatment process cost-effectively with high quality.

### SUMMARY

Disclosed herein are intelligent methods or systems for process and asset health diagnosis and anomaly detection in wastewater treatment plants or drinking water plants. The system includes the entire diagnosis methodology to determine the plant health status including process and asset health. The results can be pushed out to a user interface as notifications or to a control system for actions taken in accordance with the results. Data for diagnosis can be obtained from one or more of influent sensors, assets sensors, process sensors, effluent sensors, lab tests, plant dynamic or static simulated model, any other models to simulate or predict the plant process or asset, and the like. Compared with traditional human experience or simple threshold method, the systems and methods described herein combine a series of advanced methods or algorithms to get more comprehensive and reliable diagnosis results. The systems and methods described herein provide an intelligent water plant diagnosis service or product to end user for better monitoring and control and management of daily operations. The algorithms or models can be, but are not limited to supervised learning, unsupervised learning, risk recognition, anomaly detection, statistical analytics, cross validation, and the like. All the algorithms or models could be continuously upgraded as data loads.

Furthermore, methods and systems are disclosed herein for dynamic control and operation of a water plant using predictive analytics with synergy of physics-based model and plant data-based models/algorithms. The water treatment plants include waste water plants and drinking water plants. Embodiments of the system acquire plant data to capture the plant dynamic features, analyze in its intelligent module of “plant health diagnosis” and “advanced controller” to predict the plant performance proactively and optimize its control and operation, and then pass the optimized control strategy to the plant lower control system for real-time control. The intelligent module is where the synergy of plant physics-based model and data-based model/algorithm lies. This intelligent control system improves the plant operation and control to the knowledge and data-based level from traditional experienced level, and it can handle much more complex situations, and make the plant control and operation more reliable and effective. The intelligent control of water treatment control can effectively utilize the plant facility based on its dynamic status, and balance the effluent quality and plant operation cost, and improve the plant productivities and reliability. Also disclosed herein is an approach or methodology to quickly solve the optimal control strategies or parameters with a certain level of safety.

Disclosed herein are embodiments of a method of intelligent water plant health diagnosis and anomaly detection comprising acquiring data from a water plant; analyzing the acquired data to make a health diagnosis or anomaly detection for the water plant; and taking one or more actions based on the health diagnosis or anomaly detection for the water plant.

In one aspect, the water plant comprises a wastewater treatment plant or a drinking water plant.

Acquiring the data from the water plant may comprise acquiring the data using one or more influent sensors, asset sensors, process sensors, effluent sensors, lab tests, plant dynamic or static simulated models, and the like.

Analyzing the acquired data to make the health diagnosis or anomaly detection for the water plant may comprise applying one or more diagnosis methodologies to the acquired data such as supervised learning, unsupervised learning, cross validation with simulated model, anomaly detection, and risk pattern recognition.

In one aspect, the supervised learning diagnosis methodology comprises a machine learning task of inferring a function from labeled training data. The training data may be obtained from a historical or online database generated from water plant sensors or simulated models. The labels may comprise one or more of plant health status, risk level, anomaly, problem, root cause, and mitigation solution. In one aspect, the supervised learning diagnosis methodology learns diagnosis rules from historical events, human experience, or simulated scenarios once they are digitalized into dataset. The supervised learning diagnosis methodology can be implemented to determine or predict plant health in daily operation. The supervised learning diagnosis methodology may include one or more of decision tree, Gradient Boosting Decision Tree (GBDT)/Gradient Boosting Decision Tree (GBRT)/Multiple Addition Regression Tree (MART), Artificial Neural Network, Convolutional Neural Network (CNN), Recurrent Neural Network (RNN), Long Short Term Memory (LSTM), Gated Recurrent Unit (GRU), Support Vector Machine including all kinds of kernel methods such as RBF, Naïve Bayesian Classification, Maximum Entropy Classification, Ensemble Learning Methods including Boosting, Adaboost, Bagging, Random Forest, Linear Regression, Logistic Regression, Gaussian Process Regression, Conditional Random Field (CRF), and Compressed Sensing methods such as Sparse Representation-based Classification (SRC), and the like.

In another aspect, the unsupervised learning diagnosis methodology comprises a machine learning task of inferring a function from unlabeled data sets. The unlabeled data sets can be obtained from a historical or online database generated from water plant sensors or simulated models. One or more of plant health status, risk level, anomaly, problem, root cause, and mitigation solution can be identified by the unsupervised learning diagnosis methodology. The unsupervised learning diagnosis methodology includes one or more of Hierarchical clustering, k-means, mean-shift, spectral clustering, Singular value decomposition (SVD), Principal Component Analysis (PCA), Robust Principal Component Analysis (RPCA), Independent Component Analysis (ICA), Non-negative Matrix Factorization)(NMF), Trend Loess Decomposition (STL), Expectation Maximization (EM), Hidden Markov Model (HMM), Gaussian Mixture Model (GMM), Auto-Encoder, Variational Auto-Encoder (VAE), Generative Adversarial Nets (GAN), Deep Belief Network (DBN), Restricted Boltzmann Machine (RBM), and Least Absolute Shrinkage and Selection Operator (LASSO), and the like.

In another aspect, the cross validation with simulated model diagnosis methodology comprises cross validation of a sensor value with a corresponding value from a simulated model's outputs or lab test results to determine sensor fraud wherein a significant gap between the sensor value and the simulated model's output or lab test results provides evidence of sensor fraud. The cross validation with simulated model diagnosis methodology is used to identify, calibrate, remove or replace sensor fraud data to ensure data quality. The sensor fraud includes and not limited to noises, outliers and drift.

In another aspect, the anomaly detection diagnosis methodology comprises an algorithm to determine an anomaly or outliers from a normal dataset, wherein the anomaly includes sensor fraud data, abnormal influent or effluent water quality, abnormal energy consumption or control parameters. Generally, this methodology is used to detect anomalies that do not exist in a training dataset and is used to identify an anomaly that has not happened before. Algorithms used in anomaly detection include one or more of Maximum-Likelihood Estimation, Kalman Filter, Trend Loess Decomposition (STL), Autoregressive Integrated Moving Average model (ARIMA), and Exponential Smoothing methods such as Holt-Winters Seasonal method, and the like.

In another aspect, the risk recognition diagnosis methodology comprises a model to determine infrequent high risk events in the water plant including sludge poisoning, sludge expansion, max plant capacity exceedance, and heavy metal poisoning. The model to determine infrequent high risk events can comprise one or more of dissolved oxygen consumption rate, air flow to dissolved oxygen response model, generated sludge health index, maximum influent tolerance model, and the like.

Alternately optionally, in the embodiments of the method described above, a plurality of the diagnosis methodologies are performed in parallel to make the health diagnosis or anomaly detection for the water plant. Similarly, a plurality of the diagnosis methodologies can be performed sequentially to make the health diagnosis or anomaly detection for the water plant.

Also alternately optionally, taking one or more actions based on the health diagnosis or anomaly detection for the water plant may comprise displaying information about the health diagnosis or anomaly detection for the water plant in a graphical user interface on a display. Alternately optionally, taking one or more actions based on the health diagnosis or anomaly detection for the water plant may comprise providing data about the health diagnosis or anomaly detection for the water plant to a control system that controls at least a portion of the water plant. The data about the health diagnosis or anomaly detection for the water plant that is provided to the control system that controls at least a portion of the water plant can be used by the control system to change at least one parameter of operation of the water plant.

Also disclosed and described herein is a system for intelligent water plant health diagnosis and anomaly detection comprising a control system comprising at least a controller and one or more data acquisition components, wherein a processor in the controller executes computer-executable instruction stored in a memory of the controller, said instructions cause the processor to acquire data from a water plant using the one or more data acquisition components; analyze the acquired data to make a health diagnosis or anomaly detection for the water plant; and take one or more actions based on the health diagnosis or anomaly detection for the water plant. The one or more data acquisition components may comprise one or more influent sensors, asset sensors, process sensors, effluent sensors, lab tests, plant dynamic or static simulated models, and the like.

In one aspect of the system, the processor in the controller executes computer-executable instruction stored in a memory of the controller to analyze the acquired data to make the health diagnosis or anomaly detection for the water plant comprises the processor in the controller executes computer-executable instruction to apply one or more diagnosis methodologies to the acquired data. The one or more diagnosis methodologies comprise one or more of supervised learning, unsupervised learning, cross validation with simulated model, anomaly detection, and risk pattern recognition.

In one aspect of the system, the supervised learning diagnosis methodology comprises a machine learning task of inferring a function from labeled training data. The training data may be obtained from a historical or online database generated from water plant sensors or simulated models. The labels may comprise one or more of plant health status, risk level, anomaly, problem, root cause, and mitigation solution. In one aspect, the supervised learning diagnosis methodology learns diagnosis rules from historical events, human experience, or simulated scenarios once they are digitalized into dataset. The supervised learning diagnosis methodology can be implemented to determine or predict plant health in daily operation. The supervised learning diagnosis methodology may include one or more of decision tree, Gradient Boosting Decision Tree (GBDT)/Gradient Boosting Decision Tree (GBRT)/Multiple Addition Regression Tree (MART), Artificial Neural Network, Convolutional Neural Network (CNN), Recurrent Neural Network (RNN), Long Short Term Memory (LSTM), Gated Recurrent Unit (GRU), Support Vector Machine including all kinds of kernel methods such as RBF, Naïve Bayesian Classification, Maximum Entropy Classification, Ensemble Learning Methods including Boosting, Adaboost, Bagging, Random Forest, Linear Regression, Logistic Regression, Gaussian Process Regression, Conditional Random Field (CRF), and Compressed Sensing methods such as Sparse Representation-based Classification (SRC), and the like.

In another aspect of the system, the unsupervised learning diagnosis methodology comprises a machine learning task of inferring a function from unlabeled data sets. The unlabeled data sets can be obtained from a historical or online database generated from water plant sensors or simulated models. One or more of plant health status, risk level, anomaly, problem, root cause, and mitigation solution can be identified by the unsupervised learning diagnosis methodology. The unsupervised learning diagnosis methodology includes one or more of Hierarchical clustering, k-means, mean-shift, spectral clustering, Singular value decomposition (SVD), Principal Component Analysis (PCA), Robust Principal Component Analysis (RPCA), Independent Component Analysis (ICA), Non-negative Matrix Factorization)(NMF), Trend Loess Decomposition (STL), Expectation Maximization (EM), Hidden Markov Model (HMM), Gaussian Mixture Model (GMM), Auto-Encoder, Variational Auto-Encoder (VAE), Generative Adversarial Nets (GAN), Deep Belief Network (DBN), Restricted Boltzmann Machine (RBM), and Least Absolute Shrinkage and Selection Operator (LASSO), and the like.

In another aspect of the system, the cross validation with simulated model diagnosis methodology comprises cross validation of a sensor value with a corresponding value from a simulated model's outputs or lab test results to determine sensor fraud wherein a significant gap between the sensor value and the simulated model's output or lab test results provides evidence of sensor fraud. The cross validation with simulated model diagnosis methodology is used to identify, calibrate, remove or replace sensor fraud data to ensure data quality.

In another aspect of the system, the anomaly detection diagnosis methodology comprises an algorithm to determine an anomaly or outliers from a normal dataset, wherein the anomaly includes sensor fraud data, abnormal influent or effluent water quality, abnormal energy consumption or control parameters. Generally, this methodology is used to detect anomalies that do not exist in a training dataset and is used to identify an anomaly that has not happened before. Algorithms used in anomaly detection include one or more of Maximum-Likelihood Estimation, Kalman Filter, Trend Loess Decomposition (STL), Autoregressive Integrated Moving Average model (ARIMA), and Exponential Smoothing methods such as Holt-Winters Seasonal method, and the like.

In another aspect of the system, the risk recognition diagnosis methodology comprises a model to determine infrequent high risk events in the water plant including sludge poisoning, sludge expansion, max plant capacity exceedance, and plant capability such as heavy metal poisoning and including water chemistry, such as heavy metal or other recalcitrant organic contaminants. The model to determine infrequent high risk events can comprise one or more of dissolved oxygen consumption rate, air flow to dissolved oxygen response model, generated sludge health index, maximum influent tolerance model, and the like.

Alternately optionally, in the embodiments of the system described above, a plurality of the diagnosis methodologies are performed in parallel to make the health diagnosis or anomaly detection for the water plant. Similarly, a plurality of the diagnosis methodologies can be performed sequentially to make the health diagnosis or anomaly detection for the water plant.

Also alternately optionally, the system further comprises a display in communication with the processor of the controller and taking one or more actions based on the health diagnosis or anomaly detection for the water plant may comprise displaying information about the health diagnosis or anomaly detection for the water plant in a graphical user interface on the display. Alternately optionally, taking one or more actions based on the health diagnosis or anomaly detection for the water plant may comprise providing data about the health diagnosis or anomaly detection for the water plant to a control system that controls at least a portion of the water plant. The data about the health diagnosis or anomaly detection for the water plant that is provided to the control system that controls at least a portion of the water plant can be used by the control system to change at least one parameter of operation of the water plant.

Additional advantages will be set forth in part in the description which follows or may be learned by practice. The advantages will be realized and attained by means of the elements and combinations particularly pointed out in the appended claims. It is to be understood that both the foregoing general description and the following detailed description are exemplary and explanatory only and are not restrictive, as claimed.

### DRAWINGS

The accompanying drawings, which are incorporated in and constitute a part of this specification, illustrate embodiments and together with the description, serve to explain the principles of the methods and systems:

FIG. 1A is an exemplary overview figure for the process of intelligent water plant health diagnosis and anomaly detection;

FIG. 1B is an example of such an integrated diagnosis module;

FIG. 1C is a flowchart illustrating an exemplary method of intelligent water plant health diagnosis and anomaly detection;

FIG. 2A is a block diagram of an exemplary wastewater treatment plant;

FIGS. 2B and 2C illustrate that diagnoses can be performed in each module in parallel and/or sequentially;

FIG. 3 is an exemplary diagnosis result;

FIGS. 4A and 4B are exemplary GUIs rendered on a display;

FIG. 5 shows the high level architecture of an intelligent control system of a water plant comprising sub-modules of “plant data acquisition,” “plant health diagnosis,” “advanced controller,” and “plant lower control system”;

FIG. 6 is a flowchart that schematically shows how the “advanced controller” works as the brain of the intelligent control system, and the “ML optimizer” and “plant operation optimization model” are coupled together as the core of the advanced controller; and

FIG. 7 illustrates an exemplary computer that can be used for performing the methods disclosed herein.

### DETAILED DESCRIPTION

Before the present methods and systems are disclosed and described, it is to be understood that the methods and systems are not limited to specific methods, specific components, or to particular compositions. It is also to be understood that the terminology used herein is for the purpose of describing particular embodiments only and is not intended to be limiting.

As used in the specification and the appended claims, the singular forms “a,” “an” and “the” include plural referents unless the context clearly dictates otherwise. Ranges may be expressed herein as from “about” one particular value, and/or to “about” another particular value. When such a range is expressed, another embodiment includes¬from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint.

“Optional” or “optionally” means that the subsequently described event or circumstance may or may not occur, and that the description includes instances where said event or circumstance occurs and instances where it does not.

Throughout the description and claims of this specification, the word “comprise” and variations of the word, such as “comprising” and “comprises,” means “including but not limited to,” and is not intended to exclude, for example, other additives, components, integers or steps. “Exemplary” means “an example of” and is not intended to convey an indication of a preferred or ideal embodiment. “Such as” is not used in a restrictive sense, but for explanatory purposes.

Disclosed are components that can be used to perform the disclosed methods and systems. These and other components are disclosed herein, and it is understood that when combinations, subsets, interactions, groups, etc. of these components are disclosed that while specific reference of each various individual and collective combinations and permutation of these may not be explicitly disclosed, each is specifically contemplated and described herein, for all methods and systems. This applies to all aspects of this application including, but not limited to, steps in disclosed methods. Thus, if there are a variety of additional steps that can be performed it is understood that each of these additional steps can be performed with any specific embodiment or combination of embodiments of the disclosed methods.

The present methods and systems may be understood more readily by reference to the following detailed description of preferred embodiments and the Examples included therein and to the Figures and their previous and following description.

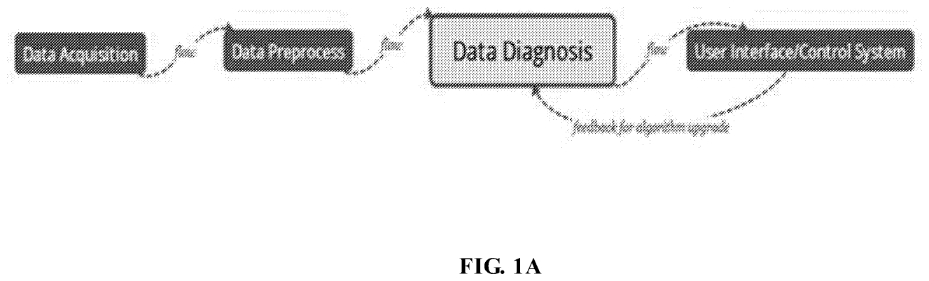

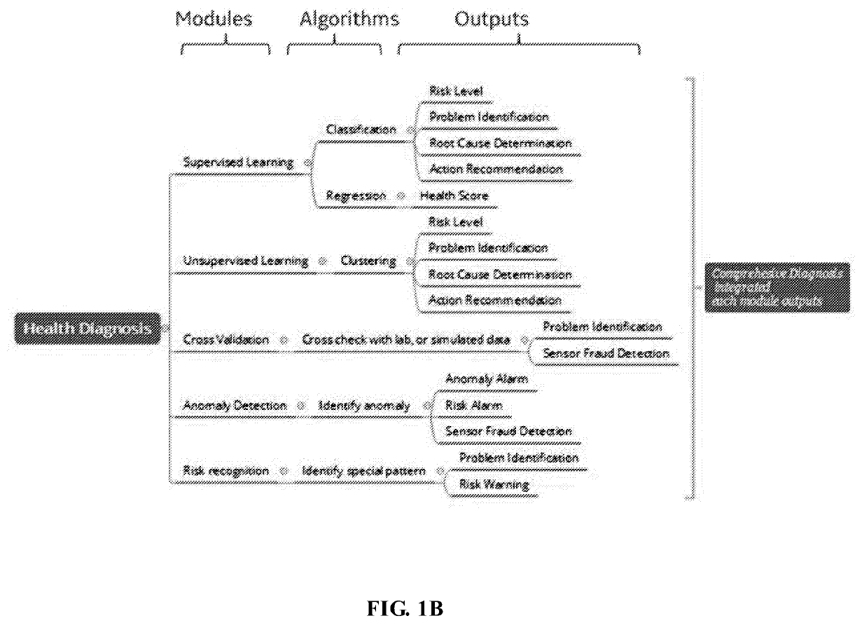

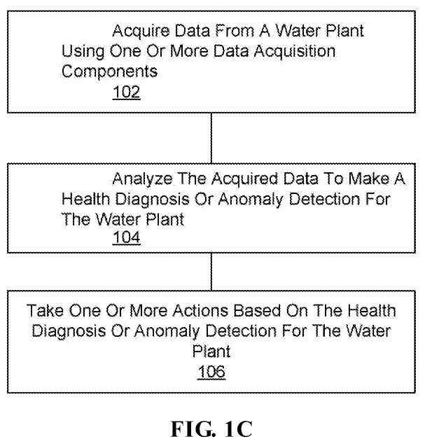

FIG. 1A is an exemplary overview figure for the process of intelligent water plant health diagnosis and anomaly detection. As illustrated in FIG. 1A, the basic process comprises data acquisition from but not limited to online sensors, lab tests, or simulated models; an option step of data preprocess to deal with bias, missing, noise or imbalance; data diagnosis by one or more algorithm packages to get more comprehensive and reliable diagnosis results. Once obtained, diagnosis results can be pushed out to user interface as notifications or to control system as actions. The algorithms or models could be continuously upgraded with feedback data or new data inputs.

The diagnosis methodologies include but are not limited to supervised learning, unsupervised learning, cross validation with simulated model, anomaly detection, risk pattern recognition, and the like. The final diagnosis results may be determined by the integrated outputs of each module. The overlapped parts of outputs could be integrated by a simple voting mechanism or a weighted voting mechanism. The final diagnosis results could include but is not limited to problem identification, risk level, root cause, recommended actions, health score, sensor fraud alarm, anomaly alarm, and the like. An example of such an integrated diagnosis module is shown in FIG. 1B.

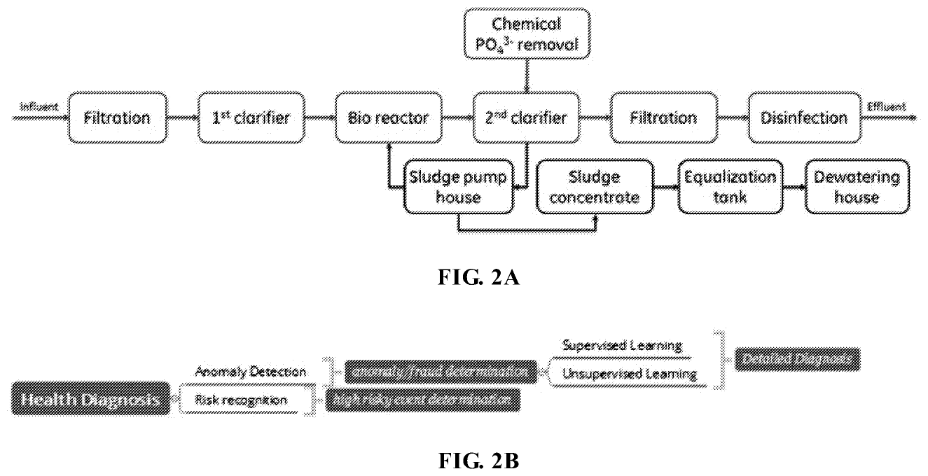

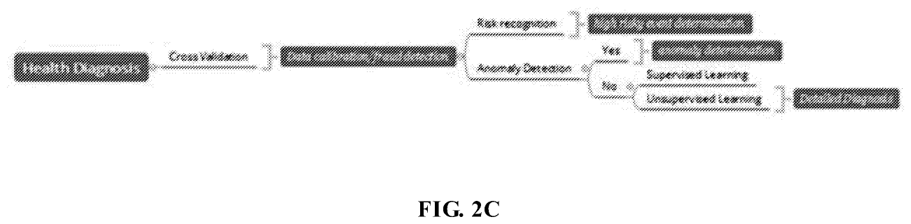

FIG. 1C is a flowchart illustrating an exemplary method of intelligent water plant health diagnosis and anomaly detection. The exemplary method comprises, at 102, acquiring data from a water plant. The water plant may comprise, for example, a wastewater treatment plant, a drinking water plant, and the like. The data may comprise data from water chemistry sensors, asset sensors, influent sensors, process sensors, effluent sensors, lab tests, plant dynamic or static simulated models, and the like. FIG. 2A is a block diagram of an exemplary wastewater treatment plant. Table I is an example list of data collected water chemistry sensors, and their location within the typical wastewater plant of FIG. 2A. Table II, below, is an example list of asset sensors and the data they collect.

TABLE I

| Sensors | Installation position |
|---|---|
| Temp. | Influent |
| Aqueous flow meter | Influent |
| pH | Influent |
| BOD | Influent |
| COD | Influent |
| Alkalinity | Influent |
| NH₃—N | Influent |
| NO₃—N | Influent |
| TSS | Influent |
| TN | Influent |
| PO₄³⁻ | Influent |
| TP | Influent |
| Gas flow meter | aerobic tank |
| DO | aerobic tank |
| NH₃—N | aerobic tank |
| NO₃—N | aerobic tank |
| MLSS | aerobic tank |
| ORP | anaerobic/anoxic tank |
| TN/NO₃—N, NO₂—N | Bioreactor effluent |
| TN | Bioreactor effluent |
| TP | Bioreactor effluent |
| Temp. | Effluent |
| Aqueous flow meter | Effluent |
| pH | Effluent |
| TSS | Effluent |
| BOD | Effluent |
| NH3—N | Effluent |
| TN | Effluent |
| TP | Effluent |

### Selected Water Chemistry Sensors in a Wastewater Treatment Plant

TABLE II

| Assets | Sensors |
|---|---|
| Air blower | temp |
| | gas flow rate |
| | pipeline pressure |
| | frequency |
| | Voltage |
| | Current |
| hydraulic pump | flow rate |
| | Pressure |
| sludge pump | flow rate |
| | pressure |

### Selected Asset Sensors in a Wastewater Treatment Plant

Returning to the flowchart of FIG. 1, at 104, the acquired data is analyzed to make a health diagnosis or anomaly detection for the water plant. At 104, the obtained sample of the hydrocarbon composition is analyzed to determine one or more attributes of the sample. Analyzing the acquired data to make the health diagnosis or anomaly detection for the water plant generally comprises applying one or more diagnosis methodologies to the acquired data. The one or more diagnosis methodologies comprise one or more of supervised learning, unsupervised learning, cross validation with simulated model, anomaly detection, risk pattern recognition, and the like, as further described below.

Supervised learning is one machine learning task of inferring a function from labeled training data. The training data can be obtained from the historical or online database generated from water plant sensors or simulated models. The labels can be the plant health status, risk level, anomaly, problem, root cause, or mitigation solution. These models learn the diagnosis rules from historical events, human experience, or simulated scenarios once they are digitalized into a dataset. Then, the models are implemented to determine or predict plant health in daily operation. The algorithms used can be one or more of Decision tree, Gradient Boosting Decision Tree (GBDT)/Gradient Boosting Decision Tree (GBRT)/Multiple Addition Regression Tree (MART), Artificial Neural Network, Convolutional Neural Network (CNN), Recurrent Neural Network (RNN), Long Short Term Memory (LSTM), Gated Recurrent Unit (GRU), Support Vector Machine including all kinds of kernel methods such as RBF, Naïve Bayesian Classification, Maximum Entropy Classification, Ensemble Learning Methods including Boosting, Adaboost, Bagging, Random Forest, Linear Regression, Logistic Regression, Gaussian Process Regression, Conditional Random Field (CRF), Compressed Sensing methods such as Sparse Representation-based Classification (SRC), and the like.

Unsupervised learning comprises using the diagnosis rules from historical or online database without labeled responses. This is a complementary method to supervised learning. More unlabeled dataset could be involved into the diagnosis than are used with supervised learning. Plant health status, risk level, anomaly, problem, root cause or mitigation solution may also be identified by unsupervised learning in some extent. The algorithms used in unsupervised learning can be one or more of Hierarchical clustering, k-means, mean-shift, spectral clustering, Singular value decomposition (SVD), Principal Component Analysis (PCA), Robust Principal Component Analysis (RPCA), Independent Component Analysis (ICA), Non-negative Matrix Factorization)(NMF), Trend Loess Decomposition (STL), Expectation Maximization (EM), Hidden Markov Model (HMM), Gaussian Mixture Model (GMM), Auto-Encoder, Variational Auto-Encoder (VAE), Generative Adversarial Nets (GAN), Deep Belief Network (DBN), Restricted Boltzmann Machine (RBM), Least Absolute Shrinkage and Selection Operator (LASSO), and the like.

Cross validation of the sensor value with the corresponding value from simulated model's outputs or lab test results is a method to determine sensor fraud. A significant gap between sensor value and simulated soft sensor or lab test results can provide evidence of sensor fraud. By using cross-validation, sensor fraud can be identified, calibrated (to correct), removed or replaced in order to ensure data quality.

Anomaly detection is a method to determine anomaly or outliers from normal dataset. The anomaly may include sensor fraud data, abnormal influent or effluent water quality, abnormal energy consumption or control parameters. The anomaly may not necessarily exist in training dataset and it is also not possible to cover all the anomaly scenarios in the training dataset. Therefore, this is a suitable method to identify an anomaly that has not happened before. The algorithms used can be one or more of Maximum-Likelihood Estimation, Kalman Filter, Trend Loess Decomposition (STL), Autoregressive Integrated Moving Average model (ARIMA), Exponential Smoothing methods such as Holt-Winters Seasonal method, and the like.

Risk recognition is a method to determine the high risk events in water plants. These kinds of events do not occur often, but require a special analysis to identify an include events such as sludge poisoning, sludge expansion, max plant capacity exceedance or heavy metal poisoning. Models are created to recognize these high risk events. The models include but are not limited to dissolved oxygen consumption rate, air flow to dissolved oxygen response model, generated sludge health index, or maximum influent tolerance model. By this way, the special pattern of high risk events can be identified for warning or problem identification.

As shown in FIGS. 2B and 2C, the diagnosis can be performed in each module in parallel and/or sequentially; or, as shown in FIG. 2C, some other logical combinations of these modules to generate the diagnosis results are also feasible. The modules could also be partially selected to generate diagnosis results. For example, in FIG. 2B, first determine high risk event and anomaly, if not, flow to detailed diagnosis by supervised/unsupervised learning. In FIG. 2C, first calibrate the data by cross validation, then flow to next level to identify high risk or anomaly, if not, flow to detailed diagnosis by supervised/unsupervised learning. It is to be appreciated the FIGS. 2B and 2C illustrate non-limiting examples.

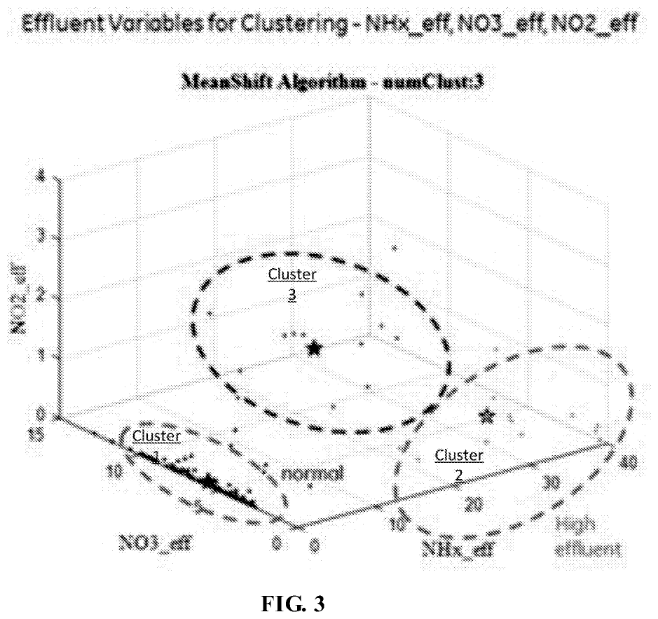

FIG. 3 is an exemplary diagnosis result that illustrates three nitrogen effluent health clusters determined by the clustering algorithm in one typical water plant; Cluster 1—normal status; Cluster 2—risky (high NHx-eff); and Cluster 3—highly risky (high NHx-eff, high NOx-eff). Table III, below, is an example of supervised learning shown diagnosis clusters vs data labels (problem identification and root cause):

TABLE III

| Cluster | Problem Identification and root cause |
|---|---|
| 1 | NHx exceedance, Incoming load exceedance |
| 2 | NHx, NO₂ exceedance, Inadequate |
| | Nitrification |
| 3 | NO₃ exceedance, Poor Nitrification |
| 4 | Approaching anomalous behavior |
| 5 | Healthy |
| 6 | NO₂ exceedance, Poor Nitrification |

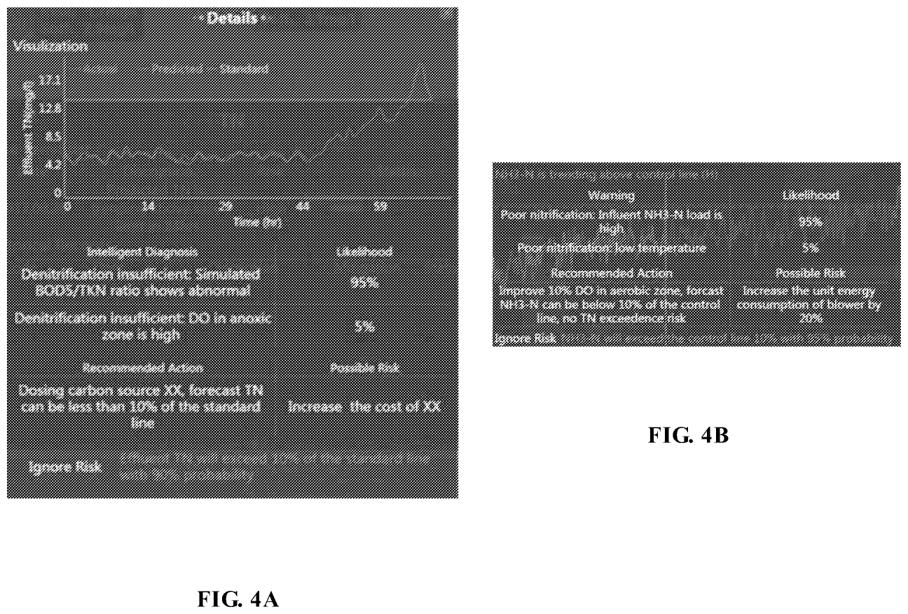

Returning to the flowchart of FIG. 1A, at 106 one or more actions are taken based on the health diagnosis or anomaly detection for the water plant. In one aspect, such actions may comprise displaying information about the health diagnosis or anomaly detection for the water plant in a graphical user interface (GUI) on a display. FIGS. 4A and 4B are exemplary GUIs rendered on a display. These exemplary diagnosis results displayed on the GUI include risk warning, problem identification, root cause, recommended actions, and the like. The information rendered can be dependent upon various criteria including who the diagnosis is sent to and that person's authority, the type of electronic device used to render the graphic, and the like. It is to be appreciated that the display can be the display of any electronic device including a computer, a laptop computer, a smart phone, a portable smart device such as an iPad™, and the like.

Alternatively or concurrently, taking one or more actions based on the health diagnosis or anomaly detection for the water plant may comprise providing data about the health diagnosis or anomaly detection for the water plant to a control system that controls at least a portion of the water plant where the data about the health diagnosis or anomaly detection for the water plant is used by the control system to change at least one parameter of operation of the water plant.

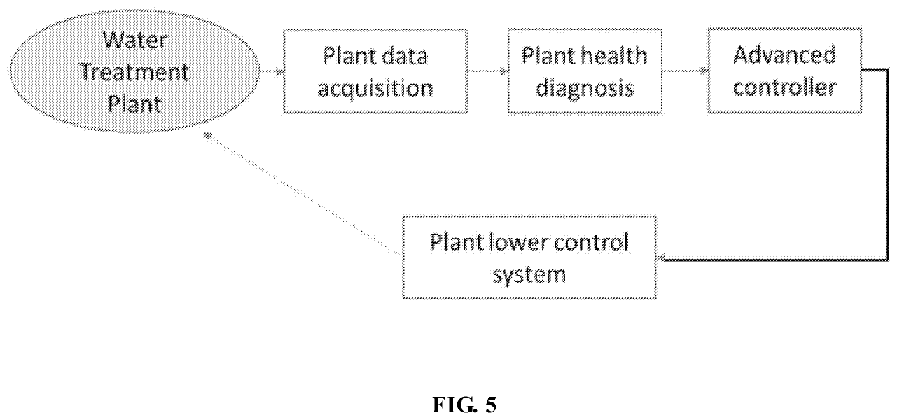

FIG. 5 shows the high level architecture of an intelligent control system of a water plant comprising sub-modules of “plant data acquisition,” “plant health diagnosis,” “advanced controller,” and “plant lower control system.” “Plant data acquisition” is to obtain the plant data and information including but not limited to historical and real-time on-line sensors, lab test, patrol inspection, and the like. Plant health diagnosis is a package of algorithms and models, as described above, to provide more comprehensive and reliable diagnostics on the plant health and determine if it's necessary to optimize the plant control operation and therefore set the constraints for the control optimization based on the diagnostics results. “Advanced controller” performs the whole plant operation optimization and obtains the optimal operation set of control parameters/strategy, and then passes them to the “plant lower control system” for implementing at the plant. “Plant lower control system” refers to the plant on-site control execution system including but not limited to SCADA, PLC, etc.

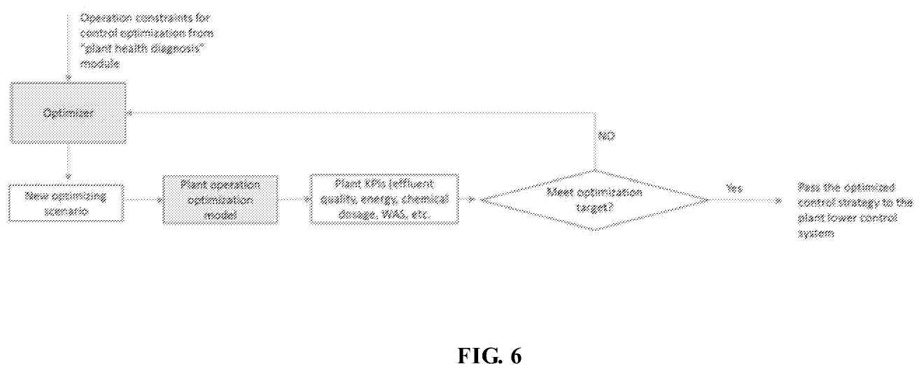

FIG. 6 is a flowchart that schematically shows how the “advanced controller” works as the brain of the intelligent control system, and the “ML optimizer” and “plant operation optimization model” are coupled together as the core of the advanced controller. The optimizer uses machine learning and artificial intelligence techniques to dynamically generate optimization scenario for the plant operation optimization model to run and validate. Once the optimization target with one scenario is met, that control strategy of that scenario will be passed to the plant lower control system to implement.

“Plant health diagnosis” model has plant design and retrofit data and information as its basic input, and it will continuously receive dynamic influent data including flowrate and quality during operation. With all these information, the plant health diagnosis module, as described above, continuously checks the plant health status and if it's necessary will perform operation optimization tasks. Once an optimization need is identified, it will trigger the “optimizer” of the advanced controller and send the operation constraints to the “optimizer”. Machine learning technique are used in the plant health diagnosis module to identify the operation constraints for control optimization based on the plant dynamic status and narrow the optimization space.

The “optimizer” is based on the machine learning technique and it enhances the resolver of the advanced controller. It integrates constraints produced from “plant health diagnosis” module, water treatment knowledge, plant data and results of previous optimizing scenario to dynamically generate next optimizing instance for the plant operation optimization model to run and estimate. This is desirable compared with existing technique with fixed pre-set scenario matrices to find optimal point in terms of total number of scenarios to run and the speed to find the optimal point.

The plant operation optimization model is a collection of models representing the biological, chemical, hydraulic, etc. features of plant units and operations. It is firstly set up based on the unit/operation mechanism/physics and then calibrated with the plant specific data and information to form the virtual copy of the plant. This enables it mimic the plant behavior and accurately monitor and predict the plant performance including key performance indicators (KPIs) once information on influent flowrate and quality is received. This module includes but is not limited to mechanistic physics-based predictive models of biokinetics like activated sludge models (ASMs), chemical dosing for alkalinity adjustment, phosphorous control, extra carbon introduction, aggregation/flocculation, settling, oxygen transfer, aeration control, pump control, etc. and their individual and overall simplified ones. The plant KPIs include but not limit to effluent quality like total suspended solids (TSS), BOD (biochemical oxygen demand), COD (chemical oxygen demand), TOC (total organic carbon) TP (total phosphorous), TN (total nitrogen), NH3-N (ammoniacal nitrogen); energy consumption/cost; chemical consumption/cost; WAS generation/deposal cost; overall cost; and the like.

The solutions presented in the present application can be conducted with a time lag, or they can be conducted dynamically, which is essentially in real-time with the use of appropriate computer processors.

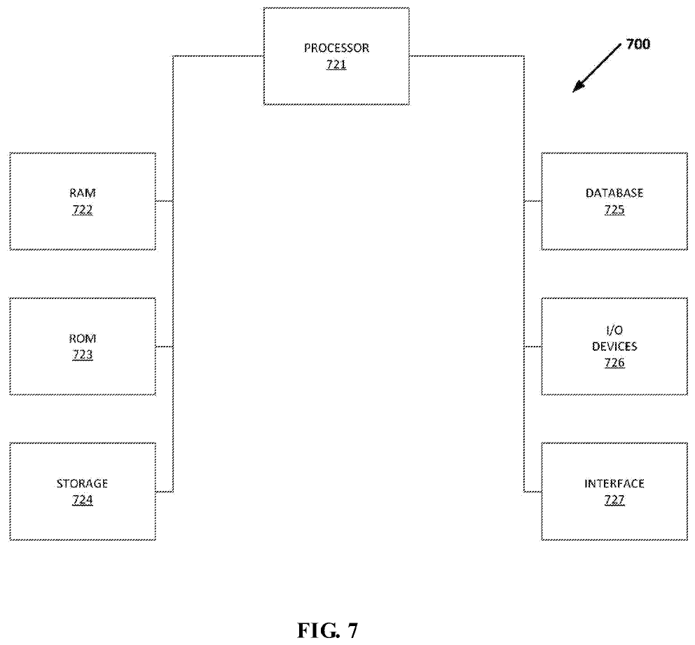

The system has been described above as comprised of units. One skilled in the art will appreciate that this is a functional description and that the respective functions can be performed by software, hardware, or a combination of software and hardware. A unit can be software, hardware, or a combination of software and hardware. The units can comprise software for intelligent water plant health diagnosis, anomaly detection and control. In one exemplary aspect, the units can comprise a controller 700 that comprises a processor 721 as illustrated in FIG. 7 and described below.

Furthermore, all or portions of aspects of the disclosed can be implemented using cloud-based processing and storage systems and capabilities. The controller 700 described in relation to FIG. 7 may comprise a portion of a cloud-based processing and storage system. One such non-limiting example of a cloud-base service that can be used in implementations of the disclosed is GE Predix™, as available from the General Electric Company (Schenectady, N.Y.). Predix™ is a cloud-based PaaS (platform as a service) that enables industrial-scale analytics for asset performance management (APM) and operations optimization by providing a standard way to connect machines, data, and people.

FIG. 7 illustrates an exemplary controller 700 that can be used for acquiring data from a water plant; analyzing the acquired data to make a health diagnosis or anomaly detection for the water plant; and taking one or more actions based on the health diagnosis or anomaly detection for the water plant. In various aspects, the computer of FIG. 7 may comprise all or a portion of the controller 700 and/or a process control system. As used herein, “controller” may comprise a computer and includes a plurality of computers. The controller 700 may include one or more hardware components such as, for example, a processor 721, a random access memory (RAM) module 722, a read-only memory (ROM) module 723, a storage 724, a database 725, one or more input/output (I/O) devices 726, and an interface 727. Alternatively and/or additionally, the controller 700 may include one or more software components such as, for example, a computer-readable medium including computer executable instructions for performing a method associated with the exemplary embodiments. It is contemplated that one or more of the hardware components listed above may be implemented using software. For example, storage 724 may include a software partition associated with one or more other hardware components. It is understood that the components listed above are exemplary only and not intended to be limiting.

Processor 721 may include one or more processors, each configured to execute instructions and process data to perform one or more functions associated with intelligent water plant health diagnosis, anomaly detection and control. As used herein, “processor” 721 refers to a physical hardware device that executes encoded instructions for performing functions on inputs and creating outputs. Processor 721 may be communicatively coupled to RAM 722, ROM 723, storage 724, database 725, I/O devices 726, and interface 727. Processor 721 may be configured to execute sequences of computer program instructions to perform various processes. The computer program instructions may be loaded into RAM 722 for execution by processor 721.

RAM 722 and ROM 723 may each include one or more devices for storing information associated with operation of processor 721. For example, ROM 723 may include a memory device configured to access and store information associated with controller 700, including information for identifying, initializing, and monitoring the operation of one or more components and subsystems. RAM 722 may include a memory device for storing data associated with one or more operations of processor 721. For example, ROM 723 may load instructions into RAM 722 for execution by processor 721.

Storage 724 may include any type of mass storage device configured to store information that processor 721 may need to perform processes consistent with the disclosed embodiments. For example, storage 724 may include one or more magnetic and/or optical disk devices, such as hard drives, CD-ROMs, DVD-ROMs, or any other type of mass media device.

Database 725 may include one or more software and/or hardware components that cooperate to store, organize, sort, filter, and/or arrange data used by controller 700 and/or processor 721. It is contemplated that database 725 may store additional and/or different information than that listed above.

I/O devices 726 may include one or more components configured to communicate information with a user associated with controller 700. For example, I/O devices 726 may include a console with an integrated keyboard and mouse to allow a user to maintain an algorithm for intelligent water plant health diagnosis, anomaly detection and control, and the like. I/O devices 726 may also include a display including a graphical user interface (GUI) for outputting information on a monitor. I/O devices 726 may also include peripheral devices such as, for example, a printer for printing information associated with controller 700, a user-accessible disk drive (e.g., a USB port, a floppy, CD-ROM, or DVD-ROM drive, etc.) to allow a user to input data stored on a portable media device, a microphone, a speaker system, or any other suitable type of interface device.

Interface 727 may include one or more components configured to transmit and receive data via a communication network, such as the Internet, a local area network, a workstation peer-to-peer network, a direct link network, a wireless network, or any other suitable communication platform. For example, interface 727 may include one or more modulators, demodulators, multiplexers, demultiplexers, network communication devices, wireless devices, antennas, modems, and any other type of device configured to enable data communication via a communication network.

While the methods and systems have been described in connection with preferred embodiments and specific examples, it is not intended that the scope be limited to the particular embodiments set forth, as the embodiments herein are intended in all respects to be illustrative rather than restrictive.

Unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is no way intended that an order be inferred, in any respect. This holds for any possible non-express basis for interpretation, including: matters of logic with respect to arrangement of steps or operational flow; plain meaning derived from grammatical organization or punctuation; the number or type of embodiments described in the specification.

Throughout this application, various publications may be referenced. The disclosures of these publications in their entireties are hereby incorporated by reference into this application in order to more fully describe the state of the art to which the methods and systems pertain.

It will be apparent to those skilled in the art that various modifications and variations can be made without departing from the scope or spirit. Other embodiments will be apparent to those skilled in the art from consideration of the specification and practice disclosed herein. It is intended that the specification and examples be considered as exemplary only, with a true scope and spirit being indicated by the following claims.

1. A method of intelligent water plant health diagnosis anomaly detection and control comprising:
acquiring data from a water plant;
analyzing the acquired data to make a health diagnosis or anomaly detection for the water plant; and
taking one or more actions based on the health diagnosis or anomaly detection for the water plant,
wherein analyzing the acquired data to make the health diagnosis or anomaly detection for the water plant comprises applying one or more diagnosis methodologies to the acquired data, wherein the one or more diagnosis methodologies comprise one or more of supervised learning, unsupervised learning, cross validation with simulated model, data driven model, anomaly detection, and risk pattern recognition.

2. (canceled)

3. (canceled)

4. (canceled)

5. (canceled)

6. The method of claim 1, wherein the supervised learning diagnosis methodology comprises a machine learning task of inferring a function from labeled training data,
wherein the supervised learning diagnosis methodology is implemented to determine or predict plant health in daily operation,
wherein the supervised learning diagnosis methodology learns diagnosis rules from historical events including both local site and global cases from a data center, human experience, or simulated scenarios once they are digitalized into dataset, and
wherein the supervised learning diagnosis methodology includes one or more of decision tree, Gradient Boosting Decision Tree (GBDT)/Gradient Boosting Decision Tree (GBRT)/Multiple Addition Regression Tree (MART), Artificial Neural Network, Convolutional Neural Network (CNN), Recurrent Neural Network (RNN), Long Short Term Memory (LSTM), Gated Recurrent Unit (GRU), Support Vector Machine including all kinds of kernel methods such as RBF, Naïve Bayesian Classification, Maximum Entropy Classification, Ensemble Learning Methods including Boosting, Adaboost, Bagging, Random Forest, Linear Regression, Logistic Regression, Gaussian Process Regression, Conditional Random Field (CRF), and Compressed Sensing methods such as Sparse Representation-based Classification (SRC).

7. (canceled)

8. (canceled)

9. (canceled)

10. (canceled)

11. (canceled)

12. The method of claim 1, wherein the unsupervised learning diagnosis methodology comprises a machine learning task of inferring a function from unlabeled data sets,
wherein one or more of plant health status, risk level, anomaly, problem, root cause, and mitigation solution are identified by the unsupervised learning diagnosis methodology,
wherein the unlabeled data sets are obtained from a historical or online database generated from water plant sensors or simulated models, and
wherein the unsupervised learning diagnosis methodology includes one or more of Hierarchical clustering, k-means, mean-shift, spectral clustering, Singular value decomposition (SVD), Principal Component Analysis (PCA), Robust Principal Component Analysis (RPCA), Independent Component Analysis (ICA), Non-negative Matrix Factorization) (NMF), Trend Loess Decomposition (STL), Expectation Maximization (EM), Hidden Markov Model (HMM), Gaussian Mixture Model (GMM), Auto-Encoder, Variational Auto-Encoder (VAE), Generative Adversarial Nets (GAN), Deep Belief Network (DBN), Restricted Boltzmann Machine (RBM), and Least Absolute Shrinkage and Selection Operator (LASSO).

13. (canceled)

14. (canceled)

15. (canceled)

16. The method of claim 1, wherein the cross validation with simulated model diagnosis methodology comprises cross validation of a sensor value with a corresponding value from a simulated model's outputs or lab test results to determine sensor fraud wherein a significant gap between the sensor value and the simulated model's output or lab test results provides evidence of sensor fraud, wherein the cross validation with simulated model diagnosis methodology is used to identify, calibrate, remove or replace sensor fraud data to ensure data quality.

17. (canceled)

18. The method of claim 1, wherein the anomaly detection diagnosis methodology comprises an algorithm to determine an anomaly or outliers from a normal dataset, wherein the anomaly includes sensor fraud data, asset risky status, abnormal influent or process water or effluent water quality, specific contaminants identification, abnormal energy consumption or abnormal chemical consumption or control parameters, wherein if the anomaly does not exist in a training dataset it is used to identify an anomaly that has not happened before, and wherein the algorithm comprises and not limited one or more of Maximum-Likelihood Estimation, Kalman Filter, Trend Loess Decomposition (STL), Autoregressive Integrated Moving Average model (ARIMA), and Exponential Smoothing methods such as Holt-Winters Seasonal method.

19. (canceled)

20. (canceled)

21. The method of claim 1, wherein the risk recognition diagnosis methodology comprises a model to determine infrequent high risk events in the water plant including contaminants detected, sludge poisoning, sludge expansion, max plant capacity exceedance, and plant capability exceedance, wherein the model to determine infrequent high risk events comprises one or more of water spectrum feature abnormal, dissolved oxygen consumption rate, air flow to dissolved oxygen response model, generated sludge health index, and maximum influent tolerance model.

22. (canceled)

23. The method of claim 1, wherein a plurality of the diagnosis methodologies are performed in parallel to make the health diagnosis or anomaly detection for the water plant, or, wherein a plurality of the diagnosis methodologies are performed sequentially to make the health diagnosis or anomaly detection for the water plant.

24. (canceled)

25. The method of claim 1, wherein taking one or more actions based on the health diagnosis or anomaly detection for the water plant comprises displaying information about the health diagnosis or anomaly detection for the water plant in a graphical user interface on a display, or
comprises providing data about the health diagnosis or anomaly detection for the water plant to a control system that controls at least a portion of the water plant, wherein the data about the health diagnosis or anomaly detection is used by the control system to change at least one parameter of operation of the water plant.

26. (canceled)

27. (canceled)

28. A system for intelligent water plant health diagnosis anomaly detection and control comprising:
a control system comprising at least a controller and one or more data acquisition components, wherein a processor in the controller executes computer-executable instruction stored in a memory of the controller, said instructions cause the processor to:
acquire data from a water plant using the one or more data acquisition components;
analyze the acquired data to make a health diagnosis or anomaly detection for the water plant by applying one or more diagnosis methodologies to the acquired data, wherein the one or more diagnosis methodologies comprise one or more of supervised learning, unsupervised learning, cross validation with simulated model, anomaly detection, and risk pattern recognition; and
take one or more actions based on the health diagnosis or anomaly detection for the water plant,
wherein the one or more data acquisition components comprise one or more local plant influent sensors, asset sensors, process sensors, effluent sensors, lab tests, plant dynamic or static simulated models, and historical data and global/cloud data base center.

29. (canceled)

30. (canceled)

31. (canceled)

32. The system of claim 28, wherein the supervised learning diagnosis methodology comprises a machine learning task of inferring a function from labeled training data,
wherein the training data is obtained from a historical or online database generated from water plant sensors or simulated models, wherein the labels comprise one or more of plant health status, risk level, anomaly, problem, root cause, and mitigation solution, wherein the supervised learning diagnosis methodology learns diagnosis rules from historical events, human experience, or simulated scenarios once they are digitalized into dataset,
wherein the supervised learning diagnosis methodology is implemented to determine or predict plant health in daily operation, and
wherein the supervised learning diagnosis methodology includes one or more of decision tree, Gradient Boosting Decision Tree (GBDT)/Gradient Boosting Decision Tree (GBRT)/Multiple Addition Regression Tree (MART), Artificial Neural Network, Convolutional Neural Network (CNN), Recurrent Neural Network (RNN), Long Short Term Memory (LSTM), Gated Recurrent Unit (GRU), Support Vector Machine including all kinds of kernel methods such as RBF, Naïve Bayesian Classification, Maximum Entropy Classification, Ensemble Learning Methods including Boosting, Adaboost, Bagging, Random Forest, Linear Regression, Logistic Regression, Gaussian Process Regression, Conditional Random Field (CRF), and Compressed Sensing methods such as Sparse Representation-based Classification (SRC).

33. (canceled)

34. (canceled)

35. (canceled)

36. (canceled)

37. (canceled)

38. The system of claim 28, wherein the unsupervised learning diagnosis methodology comprises a machine learning task of inferring a function from unlabeled data sets,
wherein the unlabeled data sets are obtained from a historical or online database generated from water plant sensors or simulated models,
wherein one or more of plant health status, risk level, anomaly, problem, root cause, and mitigation solution are identified by the unsupervised learning diagnosis methodology, and
wherein the unsupervised learning diagnosis methodology includes one or more of Hierarchical clustering, k-means, mean-shift, spectral clustering, Singular value decomposition (SVD), Principal Component Analysis (PCA), Robust Principal Component Analysis (RPCA), Independent Component Analysis (ICA), Non-negative Matrix Factorization)(NMF), Trend Loess Decomposition (STL), Expectation Maximization (EM), Hidden Markov Model (HMM), Gaussian Mixture Model (GMM), Auto-Encoder, Variational Auto-Encoder (VAE), Generative Adversarial Nets (GAN), Deep Belief Network (DBN), Restricted Boltzmann Machine (RBM), and Least Absolute Shrinkage and Selection Operator (LASSO).

39. (canceled)

40. (canceled)

41. (canceled)

42. The system of claim 28, wherein the cross validation with simulated model diagnosis methodology comprises cross validation of a sensor value with a corresponding value from a simulated model's outputs or lab test results to determine sensor fraud wherein a significant gap between the sensor value and the simulated model's output or lab test results provides evidence of sensor fraud,
wherein the cross validation with simulated model diagnosis methodology is used to identify, calibrate, remove or replace sensor fraud data to ensure data quality, wherein the anomaly detection diagnosis methodology comprises an algorithm executed by the processor to determine an anomaly or outliers from a normal dataset,
wherein the anomaly includes sensor fraud data, abnormal influent or effluent water quality, abnormal energy consumption or control parameters,
wherein if the anomaly does not exist in a training dataset it is used to identify an anomaly that has not happened before, and
wherein the algorithm executed by the processor comprises one or more of Maximum-Likelihood Estimation, Kalman Filter, Trend Loess Decomposition (STL), Autoregressive Integrated Moving Average model (ARIMA), and Exponential Smoothing methods such as Holt-Winters Seasonal method.

43. (canceled)

44. (canceled)

45. (canceled)

46. (canceled)

47. The system of claim 28, wherein the risk recognition diagnosis methodology comprises a model developed using the data by the processor to determine infrequent high risk events in the water plant including sludge poisoning, sludge expansion, max plant capacity exceedance, and heavy metal poisoning, wherein the model to determine infrequent high risk events comprises one or more of dissolved oxygen consumption rate, air flow to dissolved oxygen response model, generated sludge health index, and maximum influent tolerance model.

48. (canceled)

49. The system of claim 28, wherein a plurality of the diagnosis methodologies are performed in parallel by the processor to make the health diagnosis or anomaly detection for the water plant, or wherein a plurality of the diagnosis methodologies are performed sequentially by the processor to make the health diagnosis or anomaly detection for the water plant.

50. (canceled)

51. The system of claim 28, further comprising a display device in communication with the processor, wherein taking one or more actions based on the health diagnosis or anomaly detection for the water plant comprises displaying information about the health diagnosis or anomaly detection for the water plant in a graphical user interface on the display device.

52. The system of claim 28, wherein taking one or more actions based on the health diagnosis or anomaly detection for the water plant comprises providing data about the health diagnosis or anomaly detection for the water plant to the control system that controls at least a portion of the water plant and the data about the health diagnosis or anomaly detection for the water plant that is provided to the control system that controls at least a portion of the water plant is used by the control system to change at least one parameter of operation of the water plant.
